(19) United States
(12) Patent Application Publication
(10) Pub. No.: US 2014/0018608 A1
(43) Pub. Date: Jan. 16, 2014

(54) Empathic Stimulation of the Parasympathetic Nervous System

(71) Applicant: Margaret Moore, Wellesley, MA (US)

(72) Inventor: Margaret Moore, Wellesley, MA (US)

(21) Appl. No.: 13/549,637

(22) Filed: Jul. 16, 2012

(51) Int. Cl. A61M 21/02

(52) U.S. Cl. 600/28

(57) ABSTRACT

A method for improving a health disorder related to, e.g., insufficient activation of the parasympathetic nervous system; the method can include pairing a subject and a partner, and having a session in which a relevant physiological parameter of the partner is transmitted to the subject, with the subject moving her own parameter in the direction of the partner's.

### BACKGROUND OF THE INVENTION

Chronic stress causes or contributes to innumerable health disorders, including chronic pain such as experienced by fibromyalgia sufferers. Overstimulation of the sympathetic (SP) branch of the nervous system (which controls the fight-or-flight response) contributes to chronic stress. As importantly, under-activation of the parasympathetic (PSP) branch of the nervous system (which controls the calming response) also contributes to stress. A number of pharmacologic and non-pharmacologic interventions have been employed in efforts to render to SP system less active and/or the PSP system more active. Non-pharmacologic interventions include biofeedback to control heart rate variability (a number of commercials apps, devices, and software programs, are available for this), meditation, and breath control. Some evidence exists that the presence of an empathetic caregiver is beneficial.

### SUMMARY OF THE INVENTION

The invention is based on the idea that the PSP system can be activated by eliciting an empathic response to another person in a subject with a stress-related health disorder such as chronic pain such as is experienced by people suffering with fibromyalgia or chronic widespread pain. By “empathic” is meant the capacity for sensing, understanding, sharing in, or participating in another person's feelings, as manifested by or participation in a physiological parameter, e.g., heart rate, breathing rate, or emotional arousal of the other person.

Accordingly, the invention features a method for improving a chronic stress or stress-related health disorder in a subject by pairing the subject (one or more; preferably one) with a person we will call a partner; the partner can include a therapist, coach, family member, doctor, or friend. The partner has means for transmitting to the subject (preferably to a screen observable to the subject) a dynamic physiologic parameter of the partner which is related to reduction of the SP response and/or activation of the PSP response. Exemplary physiologic parameters include heart rate, heart rate variability, breathing rate, electrocardiogram waves, and brain waves (EEG waves).

The partner and subject have one or more remote, real-time sessions, or the subject is sent recordings of real-time sessions (less preferable) during which the partner's parameter information is transmitted to the subject such that the subject's own parameter empathically moves in the direction of the partner's such that the SP response is reduced and/or PSP activation is increased, thereby improving the chronic stress or stress-related health disorder.

In one embodiment, the physiologic parameter is heart rate, and the partner's heart rate is displayed on a screen observable to the subject; and prior to or during the session the subject is asked to empathize with the partner.

### DETAILED DESCRIPTION

### Example

A subject presents with fibromyalgia, a disorder characterized by widespread pain and which is also frequently co-morbid with anxiety, depression, and/or sleep disorder. The objective, according to the invention, is to have the sufferer herself feel empathy for another person, the partner, in this case a wellness coach, and in so doing activate her PSP system, thus reducing stress, pain, and other symptoms of fibromyalgia.

The subject and coach agree on a time for a remote stress-reduction session, and prior to the session the coach may do breathing or mindfulness exercises to arrive at a calm place physiologically prior to the session. The coach is connected (by conventional means or by a commercially available app in a digital camera) to heart rate measuring means and data transmission means (e.g., telephone line or Internet) such that a dynamic image of the partner's heart rate is transmitted to a screen observable to the subject. The partner may also have a camera dynamically or statically capture her face, which image can be transmitted to a screen observable to the subject. The system can also include audio transmission means enabling the partner and the subject to communicate by voice.

Technology exists to use a computer or an iPhone to measure heart rate. HeartMath technology uses an ear sensor to measure heart rate and heart rate variability, which can be displayed as real-time data on a computer screen. A computer app (Philips) uses video to measure changes in face color to measure heart rate, and detects shoulder rises and falls to measure breathing rate. The Stress Check app allows the user to place her finger over a digital video camera aperture whereby heart rate is measured.

Transmission of the coach's heart rate, heart rate variability, or breathing rate can be accomplished by web conferencing services wherein one's computer screen, and manipulations thereof, as well as voice, video chat, and text-based messages can be shared real-time with multiple computers.

At the outset of the session, the subject is asked, using whatever wording the coach deems appropriate, to empathize with the coach during the session, by moving her heart rate or breathing rate in the direction of the coach's, as displayed on the subject's screen. Preferably the coach's heart rate is displayed on the subject's screen as a dynamic image of a beating heart. Alternatively, the dynamic beating heart data of the coach can be transmitted in audio form, e.g., with beeps signifying beats, as in fetal heart monitors.

The session proceeds silently or with calming music for a duration of between five seconds and sixty minutes, with the coach (if there is audio) occasionally speaking words of encouragement to the subject, or singing or playing calming music. Session frequency is variable to meet the subject's needs, and can be held several times per day, daily, weekly, or every few days.

### Other Embodiments

Other embodiments are within the following claims. The method of the invention can be used to treat chronic stress or any health disorder with a stress component in which the SP response is usefully reduced and/or PSP activation usefully is increased. In addition to the health disorders recited above, the invention can be used to treat other causes of pain or stress-related illness, such as peripheral neuropathy, cancer, headache, injury, migraine, hypertension, and arthritis. The method can also be used to treat stress-related anxiety, panic attacks, depression, and other psychiatric disorders. In addition, the method can be used to build empathy in people whose behavior influences others, e.g., managers whose efficacy may be compromised by their own stress.

What is claimed is:

1. A method of improving a chronic stress or stress-related health disorder in one or more subjects, comprising
a. Pairing the subject(s) with a partner having means for transmitting to the subject a dynamic physiological parameter of the partner which is related to the reduction of the activation of the sympathetic nervous system or stress response and/or activation of the parasympathetic nervous system, and
b. Having a remote partner-subject session in which the partner's physiological parameter, or a recording thereof, is transmitted to the subject such that the same parameter in the subject moves in direction of conformity with the partner's parameter such that the chronic stress or stress-related health disorder is improved.

2. The method of claim 1, wherein the physiological parameter is a heart parameter.

3. The method of claim 1, wherein the partner's heart parameter is displayed on a screen observable to the subject(s).

4. The method of claim 3, wherein said parameter is heart rate.

5. The method of claim 3, wherein the heart parameter is displayed on the subject's screen as a dynamic graph.

6. The method of claim 2, wherein said heart parameter is displayed as an animation of a beating heart.

7. The method of claim 1, wherein, prior to or during the session, the subject is asked to empathize with the partner.

8. The method of claim of claim 1, wherein said health disorder involves pain, which is reduced by said method.

9. The method of claim 2, wherein the parameter is heart rate variability.

10. The method of claim 8, wherein the pain is caused by fibromyalgia.

11. The method of claim 8, wherein the pain is widespread chronic pain.

12. The method of claim 1, wherein the screen observable to the subject displays the real-time face of the partner, and wherein the means for displaying the parameter includes audio-visual means allowing audio communication between the subject and the partner.

13. The method of claim 1, wherein the physiological parameter is breathing rate.
